(19) United States
(12) Patent Application Publication
(10) Pub. No.: US 2011/0021869 A1
(43) Pub. Date: Jan. 27, 2011

(54) SINGLE-INCISION MINIMALLY-INVASIVE SURGICAL REPAIR OF PELVIC ORGAN/VAGINAL PROLAPSE CONDITIONS

(76) Inventor: Hilary John Cholhan, Rochester, NY (US)

Correspondence Address: Stephen B. Salai, Esq.;Harter Secrest & Emery LLP, 1600 Bausch & Lomb Place, Rochester, NY, 14604-2711, US

(21) Appl. No.: 12/509,192

(22) Filed: Jul. 24, 2009

(51) Int. Cl. A61F 2/02

(52) U.S. Cl. 600/37

(57) ABSTRACT

A system includes surgical instruments, implantable articles and procedural steps for stabilizing, introducing, delivering and affixing an absorbable or permanent biocompatible implantable graft into a patient. The instruments, implantable articles and procedural steps can be employed in transvaginal pelvic reconstructive applications to re-suspend the anterior, posterior and/or apical vaginal compartments including the bladder, rectum, bowels (i.e., enterocele) and vaginal apex with or without a uterus in situ. The surgical instruments can be incorporated into a surgical kit having an implantable graft, a graft-retaining clamp, a tacker guide, a tacker device, an affixing anchor, a retractor and a fenestrated spatula.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A “SEQUENCE LISTING”

Not applicable.

### BACKGROUND OF THE INVENTION

1. Field of the Invention

The field of the invention generally relates to surgical instruments, implantable articles and procedural steps for stabilizing, introducing, delivering and affixing an absorbable or permanent biocompatible implantable graft into a patient. In one configuration, the field of the invention includes transvaginal pelvic reconstructive applications to re-suspend the anterior, posterior and/or apical vaginal compartments including the bladder, rectum, bowels (i.e., enterocele) and vaginal apex with or without a uterus in situ.

2. Description of Related Art

Prolapse of organs, tissues and structures into and through the vaginal opening is a common malady in women. Prolapse of the uterus, bladder, rectum and/or bowels (i.e., uterine descensus/prolapse, cystocele, rectocele, enterocele) is a significant and ever-increasing health concern worldwide. It is estimated that approximately 30% to 50% of women are affected. The prevalence of pelvic organ prolapse (POP) increases with age. As life expectancy continues to increase worldwide, we can expect that POP will continue to increase in prevalence and will require therapeutic management. Multiple factors are involved in the development of POP, including pregnancy, vaginal childbirth, age-related loss of tissue elasticity and its subsequent deterioration, lifestyle choices (e.g., chronic straining/lifting, smoking).

Although non-surgical therapeutic approaches (pessary, pelvic floor muscle rehabilitation with/without estrogen supplementation) can afford temporizing relief and improvement of symptomatic POP conditions, surgical therapy offers the potential for cure. It has been estimated that the lifetime risk of surgery for POP or urinary incontinence by the age of 80 years is 11%, and this number will continue to rise for the above-mentioned reason.

Over the past 100-plus years, myriad surgical procedures and facilitating instruments have been described in addressing POP. These procedures have been accomplished using transvaginal, transabdominal and/or combined approaches. The traditional repairs involved using a combination of the endogenous tissue(s) of the patient and suture material with or without trimming excess or stretched vaginal tissue. Unfortunately, the rate of prolapse recurrence after surgical treatment was reported to be as high as 40%. As is known to those skilled in the art, the treatment of vaginal wall prolapse has been hampered by high failure rates. (There are multiple reasons for recurrence of POP including inherent weakness of the tissues and/or the structures being approximated and/or the inability of the repair site(s) to withstand the forces applied during straining and resultant increases in intra-abdominal pressures).

Over the past decade, a host of approaches have been described and evolved with the goal of developing a more durable repair. Site-specific defects are repaired and buttressed with absorbable or permanent grafts affixed to the repair site by sutures. This approach has its limitations: it is onerous, time-consuming, and spatial constraints in the operative field may preclude appropriate anchorage of the graft in situ.

Since 2005, polypropylene-based “mesh kits” have been used to address reproducibility and durability. Heretofore, two distinct approaches have been developed: an external “pulley” system and an internal “pulley” system. The former, represented by Prolift™ (Gynecare, Ethicon, Johnson & Johnson, Inc.), Apogee/Perigee™ (American Medical Systems, Inc.) and Avaulta Solo/PLUS™ (Bard, Inc.) involve external passage of retrieving trocars. Blind passage of these sharp trocars through deep recesses in the pelvis predisposes to injury of the bladder, rectum as well as other pelvic organs and neurovascular structures. The latter system, represented by Pinnacle™ (Boston Scientific Corporation, Inc.) involves transvaginal internal passage of polypropylene mesh arms using a specially-designed retrieval system (i.e., Capio device®).

Although these systems are effective, they do require a high degree of surgical aptitude and skill. Some surgeons are slow to adopt promising new surgical techniques for treating POP for a variety of reasons. Some are untrained, lack experience, find the new procedures to be too intricate, etc. For those surgeons with less-than-exceptional skills, the aforementioned approaches can be challenging and daunting. Therefore, there is an apparent need to devise a procedure and attendant instruments to allow a greater number of surgeons to perform these reconstructive procedures with greater simplicity and ease, reproducibility, long-term durability and, above all, safety. Furthermore, it must permit a more shallow learning curve.

### BRIEF SUMMARY OF THE INVENTION

The present disclosure provides surgical instruments, implantable articles and procedural steps for stabilizing, introducing, delivering and affixing an absorbable or permanent biocompatible implantable graft into a patient. In one configuration, the disclosure provides for transvaginal pelvic reconstructive applications to resuspend the anterior, posterior and/or apical vaginal compartments including the bladder, rectum, bowels (i.e., enterocele) and vaginal apex with or without a uterus in situ.

Surgical kits are disclosed for use in surgical procedures for stabilizing, introducing, delivering and affixing an absorbable or permanent biocompatible implantable graft into a patient. In one configuration, the surgical kit provides for treating POP conditions and improved surgical procedures for treating such POP conditions. The surgical kit can include an implantable graft, a graft-retaining clamp, a tacker guide, a tacker device, an affixing anchor, a retractor and a fenestrated spatula. Alternative surgical kits can include at least any two of the implantable graft, the graft-retaining clamp, the tacker guide, the tacker device, the affixing anchor, the retractor and the fenestrated spatula.

A procedure is provided for stabilizing, introducing, delivering and affixing an absorbable or permanent biocompatible implantable graft to a predetermined anatomic location. In one configuration the procedure provides for stabilizing, introducing, delivering and affixing an absorbable or permanent biocompatible implantable graft through the vagina to provide support to the anterior, posterior and/or apical compartments within the vagina in order to further provide support to the bladder, rectum, uterus and/or bowel, respectively.

In one configuration, the procedure includes passing an affixing anchor through a pre-shaped finger pocket of the implantable graft into a specific predetermined anatomic location in the patient, such as the sacro-spinous ligament-coccygeus muscle complex, the iliococcygeus muscle and the proximal portion of the arcus tendineus fasciae pelvis near the ischial spine), the finger pocket being integral with a securement appendage of the implantable graft, wherein the securement appendage can extend from a main body of the implantable graft and the finger pocket being of adequate size to receive a fingertip of the operator. Thus, the procedure can provide for the anterior and/or apical vaginal compartment suspension(s), including the uterus.

It is further contemplated the finger pocket, the securement appendage and the main body can be integrally formed. In addition, the affixing devices or anchors can be any of a variety of shapes, such as spiral, and can be absorbable or permanent.

These and other aspects and features of the present invention may be understood with reference to the following description and attached figures in which preferred embodiments of the present invention are described and illustrated.

### BRIEF DESCRIPTION OF THE SEVERAL VIEWS OF THE DRAWING(S)

FIG. 1A is a perspective view of a surgical kit having a graft-retaining clamp, a tacker guide, a tacker device, an affixing anchor, a retractor and a fenestrated spatula.

FIG. 1B is an enlarged perspective view of the area labeled FIG. 1B in FIG. 1A showing a traction surface on the graft-retaining clamp.

FIG. 1C is an enlarged perspective view of the area labeled FIG. 1C in FIG. 1A showing projections on a distal end of the tacker guide.

FIG. 2A is perspective view of an implantable graft including a pair of securement appendages, with each securement appendage having a finger pocket.

FIG. 2B is perspective view of a second configuration of the implantable graft.

FIG. 3 is a perspective view of the implantable graft in the graft-retaining clamp generally located relative to the an anatomic structure with a fingertip of the surgeon in the finger pocket, and the fingertip pressing the closed end of the finger pocket against the anatomic location to which the graft is to be secured.

FIG. 4 is the perspective view of FIG. 3 with a fingertip of the surgeon and a portion of the tacker guide in the finger pocket, with the finger tip and the tacker guide pressing the closed end of the finger pocket against the anatomic location to which the graft is to be secured.

FIG. 5 is the perspective view of FIG. 4 with a finger of the surgeon pulled back from the closed end of the finger pocket and a tacker device extended through the tacker guide to locate an affixing anchor through the closed end of the finger pocket and into the anatomic structure.

FIG. 6 is the perspective view of FIG. 5 with the surgeon maintaining pressure on the closed end of the finger pocket with the tacker guide and operably locating the tacker device in the finger pocket.

FIG. 7 is the perspective view of FIG. 6 with the tacker device and tacker guide withdrawn, and the retractor smoothing the main body of the implantable graft.

FIG. 8 is the perspective view of FIG. 7 with the fenestrated spatula supporting an edge of the main body and the end of the tacker device extending through one of the fenestrations.

### DETAILED DESCRIPTION OF THE INVENTION

The present disclosure is directed to surgical instruments and implantable articles, which can be provided in kit form and the procedural steps for stabilizing, introducing, delivering and affixing an absorbable or permanent biocompatible implantable graft into a patient. In one configuration, the present disclosure is directed to surgical instruments and implantable articles, which can be provided in kit form and the procedural steps for transvaginal pelvic reconstruction.

By the term “implantable” it is understood to include any subepithelial location of the graft. Further, although the specific configuration is set forth as a transvaginal pelvic reconstruction, it is understood the disclosed surgical kit and procedures it not limited to such specific use and can be equally employed for stabilizing, introducing, delivering and affixing an absorbable or permanent biocompatible implantable graft into a patient.

By the term “biocompatible” it is understood to mean compatible with living cells, tissues, organs, or systems.

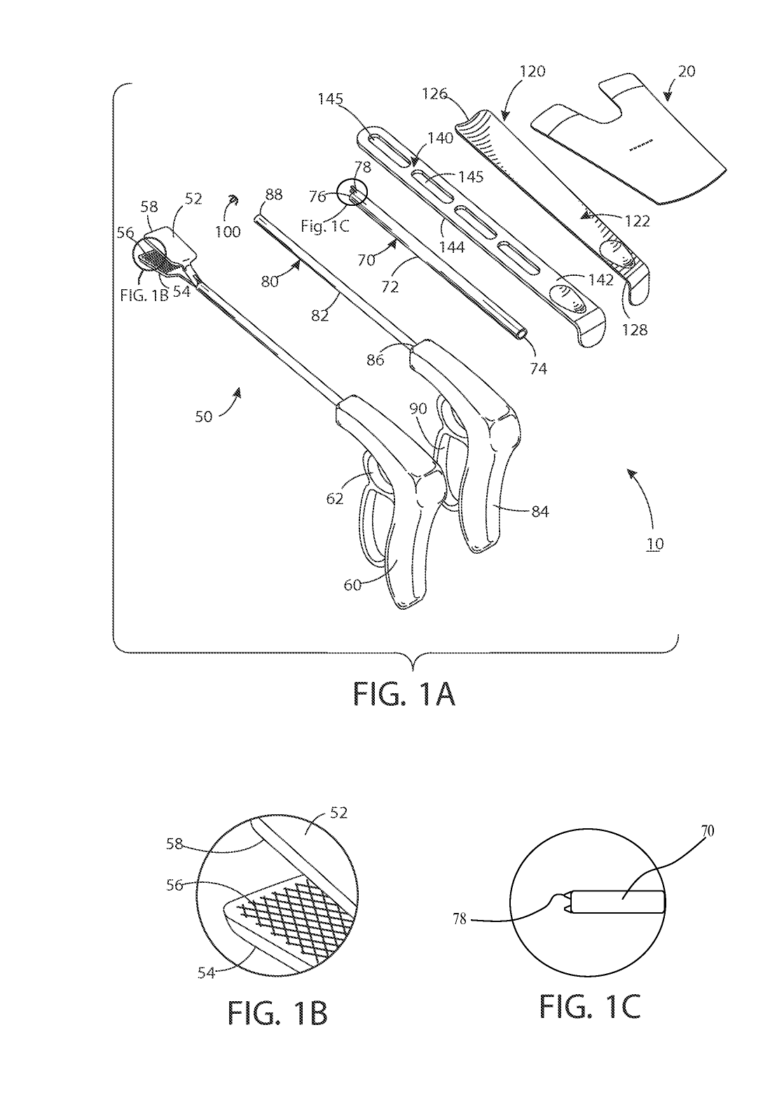

In one configuration, a surgical kit 10 is provided as seen in FIG. 1. The surgical kit 10 includes an implantable graft 20; a graft-retaining clamp 50, a tacker guide 70, a tacker device 80, an affixing anchor 100, a retractor 120 and a fenestrated spatula 140. Alternative surgical kits 10 can include at least any two of the implantable graft 20; the graft-retaining clamp 50, the tacker guide 70, the tacker device 80, the affixing anchor 100, the retractor 120 and the fenestrated spatula 140.

Generally, the implantable graft 20 is a biocompatible implantable graft, either delayed absorbable or permanent, having a predetermined or precut shape and dimensions to accommodate the anatomic relationships with the intended operable locations, such as the dimensions within the anterior, apical and/or posterior vaginal compartments. In selected configurations, the shape and dimensions of the implantable graft 20 are selected to provide for implantation in each of the three vaginal compartments separately or as a composite fused shape that will permit single implantation in order to provide simultaneous support to all three vaginal compartments. The implantable graft 20, if partially or wholly non-absorbable (permanent), can be comprised of one or more woven, knitted or inter-linked filaments or fibers that form multiple fiber junctions throughout the graft thus providing a mesh, wherein the shape and/or junctions may be formed by weaving, knitting, braiding, bonding, heat or ultrasonic bonding/welding or other such comparable techniques or combinations thereof to achieve required graft shape and/or dimensions.

The implantable graft 20 includes a main body 22 and a finger pocket 26, wherein in selected configurations the finger pocket is located on an extending securement appendage 36. In one configuration, the implantable graft 20 includes a first and a second finger pocket 26a, 26b, wherein each of the first and second finger pocket is formed on a corresponding first and second securement appendage 36a, 36b.

The main body 22 has a major dimension and a perpendicular minor dimension. For purposes of description, the major dimension is defined as extending along a longitudinal axis LA.

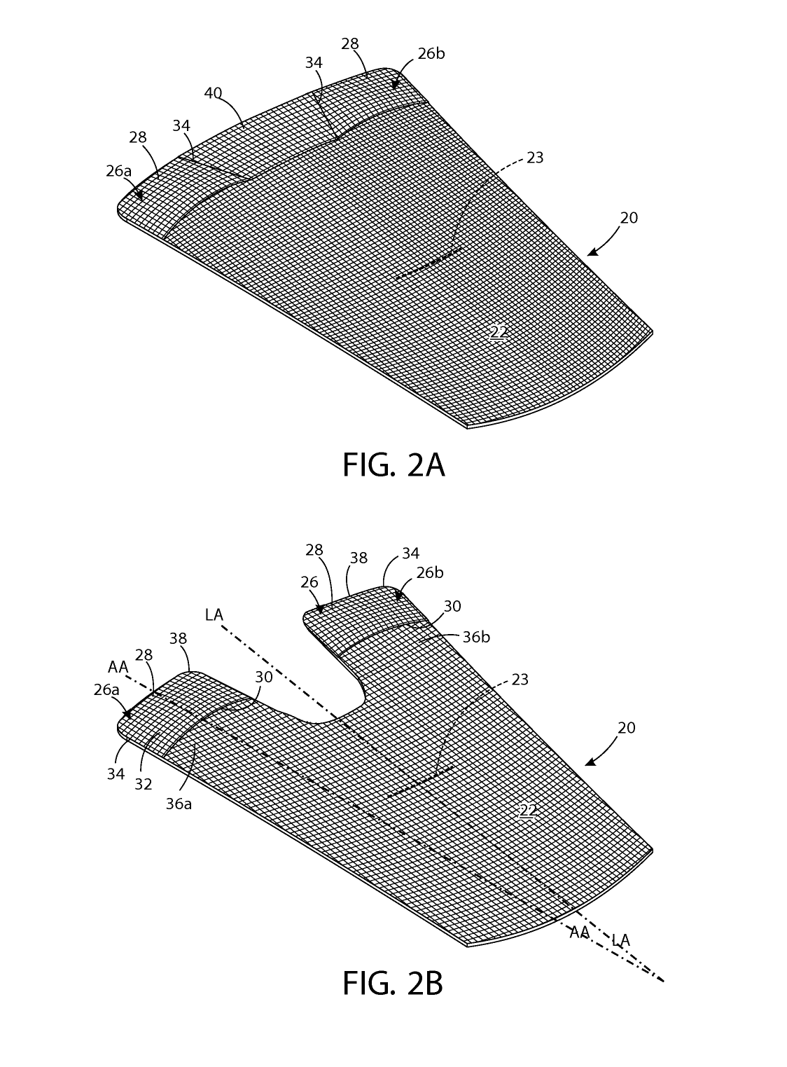

Referring to FIG. 2a, the main body 22 can include a slit or aperture 23 sized to pass a portion of the graft-retaining clamp 50. The slit 23 is located to permit a sufficient length of the graft-retaining clamp 50 to pass through the slit so that the clamp can operably engage a leading edge of the graft 20.

The finger pocket 26 has a closed end 28 and an open end 30, wherein in one configuration, the closed end is proximal to a free end 38 of a corresponding securement appendage 36 and the open end opens toward main body. The open end 30 can be transverse to an appendage axis AA or oblique to the appendage axis. The finger pocket 26 is sized to receive a finger tip, such as the distal phalange of the respective operator finger, as well as a portion of the tacker guide 70 or the tacker device 80.

While the sizing of the finger pocket 26 can be customized or unique to the given operator, it is contemplated the open end 30 of the finger pocket 26 can form a cross sectional area of approximately 12 cm². The finger pocket 26 is sized to have a depth of approximately 2 cm to approximately 3 cm. That is, at least approximately 2 cm to approximately 3 cm of the finger can be disposed within the finger pocket 26. The width of the finger pocket 26 is selected to permit simultaneous occupation by both the fingertip of the operator and a portion of the tacker guide 70. Typical sizing of the width of the finger pocket 26 is between approximately 2 cm to 3 cm.

As seen in FIG. 2a, the securement appendage 36 extends from the main body 22 along the appendage axis AA, such that in one configuration the appendage axis intersects the longitudinal axis LA. The angle of intersection between the longitudinal axis LA and the appendage axis AA can be from approximately 5° to approximately 90°. The angle between the longitudinal axis LA and the appendage axis AA is at least partly determined by the intended function of the implantable graft 20 and the available attachment or anchor locations in the patient. That is, it is contemplated the appendage axis can be parallel to the longitudinal axis.

The securement appendage 36 extends from the main body 22 to terminate at the free end 38. The securement appendage 36 can have any of a variety of profiles, such as parallel sided, tapered, bulbous or flared.

It is further understood that depending upon the intended operable location of the graft 20, the securement appendage 36 can extend from the main body 22, be substantially integrated into the main body or not be a portion of the graft. That is, the securement appendage 36 can be coterminous with a periphery of the main body 22 or extend from the adjacent portion of the periphery of the main body.

At least one, and in selected configurations each securement appendage 36a, 36b includes one finger pocket 26. Although the finger pocket 26 is shown at the free end 38 of the securement appendage 36, it is understood the finger pocket can be disposed at a location on the securement appendage intermediate the main body 22 and the free end.

For example, in one construction, the finger pockets 26 are formed by folding the material of the implantable graft 20 along a fold line as shown in FIG. 2b, to form a folded over portion 32 wherein seams 34 are formed in the folded over portion to define two spaced finger pockets. In this construction, it is contemplated that a central pocket 40 can be formed between the spaced finger pockets 26a, 26b, wherein the central pocket is sized to slidably receive a portion of the graft-retaining clamp 50.

In one configuration, it is contemplated that the main body 22, the finger pocket 26 and the securement appendage 36 can be integrally formed from a single unitary piece of material. In such construction, the section of material forming the securement appendage 36 includes an extending portion that is folded back over the securement appendage and secured along the edges to form seams 34 and thus define the finger pocket 26. Alternatively, it is contemplated that any combination of the main body 22, the finger pocket 26 and the securement appendage 36 can be formed from independent pieces of material and connected by any of a variety of known mechanisms such as stitching, bonding, fusing or gluing.

The implantable graft 20 is a biocompatible implantable graft (i.e., delayed absorbable or permanent) of a predetermined or precut shape and dimensions to accommodate the anatomic relationships and dimensions in the intended operable position. For example, the implantable graft 20 can be configured to accommodate the anatomic relationship and dimensions to fit within the anterior, apical and/or posterior vaginal compartments. The shapes and dimensions of the implantable graft 20 are intended either for implantation in each of the three vaginal compartments separately or as a composite fused shape that will permit single implantation in order to provide simultaneous support to all three vaginal compartments. The implantable graft 20, can be constructed such that the main body includes an anterior portion and a posterior portion separated by a fold line.

The material of the implantable graft 20, if partially or wholly non-absorbable/permanent, may be comprised of one or more woven, knitted or inter-linked filaments or fibers that form multiple fiber junctions throughout the mesh; the shape and/or junctions may be formed by weaving, knitting, braiding, bonding, heat or ultrasonic bonding/welding or other such comparable techniques or combinations thereof to achieve required graft shape and/or dimensions.

A suitable material for the implantable graft 20 is Gynecare's GYNEMESH™ PS, which is a knitted polypropylene mesh manufactured by Ethicon, Inc., of Somerville, N.J. This material was approved by the FDA in the United States for implantation into the human body for a variety of uses.

However, it is understood the material of the implantable graft 20 or a portion of the implantable graft can include a synthetic material or a biomaterial, or non-synthetic material. In one configuration, one portion of the implantable graft 20 is formed of an absorbable material and different second portion of the implantable graft is formed of a non-absorbable or permanent material. Thus, one portion of the implantable graft 20 can be resorbable or absorbable, a second portion may be non-absorbable and a third portion may be constructed of a different material. The implantable graft 20 can include interwoven absorbable and non-absorbable fibers or threads, such that upon absorption of the absorbable threads, the remaining non-absorbable threads form a lattice in place of the graft. It is also contemplated that a naturally occurring biomaterial may be used or a tissue engineered material may be used.

The difference in the materials for the implantable graft 20 can result in a difference in a feature that can potentially impact a surgical procedure, including the efficacy and/or results of the procedure. Features that can be different include, but are not limited to, the ability to avoid infections or tissue erosion (actual or perceived), the shelf life of the material, the type of material, the shape of the material, material treatment (e.g. heat set) or coating, the porosity of the material, the strength of the material, the elastic property of the material, the potential for tissue ingrowth and the biocompatibility of the material. Examples of treatments or coatings include anti-microbials, anti-biotics or other drug coatings.

Suitable non-synthetic materials include allografts, homografts, heterografts, autologous tissues, cadaveric fascia, autodermal grafts, dermal collagen grafts, autofascial heterografts, whole skin grafts, porcine dermal collagen, lyophilized aortic homografts, preserved dural homografts, bovine pericardium and fascia lata.

Commercial examples of non-absorbable materials include Marlex™ (polypropylene) available from Bard of Covington, R.I., Prolene™ (polypropylene), Prolene Soft Polypropylene Mesh (nonabsorbable synthetic surgical mesh) available from Ethicon, of N.J., and Mersilene (polyethylene terphthalate) Hernia Mesh also available from Ethicon. Commercial examples of absorbable materials include Dexon™ (polyglycolic acid) available from Davis and Geck of Danbury, Conn., and Vicryl™ available from Ethicon. More specific examples of synthetic materials include, but are not limited to polypropylene, cellulose, polyvinyl, polygalactin, carbon-fiber, polyethylene, nylon, polyester (e.g. Dacron™) polyanhydrides, polycaprolactone, polyglycolic acid, poly-L-lactic acid, poly-D-L-lactic acid and polyphosphate esters.

The graft-retaining clamp 50 provides for the temporary retention and stabilization of the implantable graft 20 prior to affixing to the patient. The graft-retaining clamp 50 can be sized to retain a substantial portion of the implantable graft 20 or retain localized portion of the implantable graft.

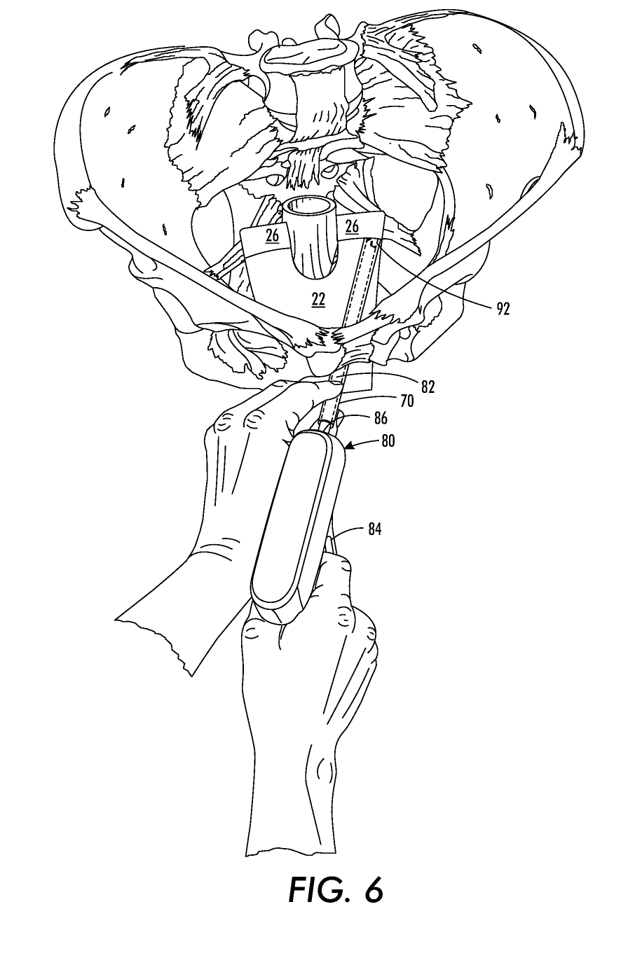

In both configurations, the graft-retaining clamp 50 includes a pair of directly opposing paddle jaws 52, 54, wherein the paddle jaws are selectively actuatable by any of a variety of mechanisms such as a handle 60 shown in FIGS. 1, 3, 6 and 7, or a pair of opposing handles with an intermediate pivot. The paddle jaws 52, 54 are moveable between an open position and a closed position. In the closed position, the graft-retaining clamp 50 stabilizes and retains the implantable graft 20.

Inner surfaces of at least one, and in selected constructions both, paddle jaws define a traction face 56 for contacting the implantable graft 20 and retaining the graft relative to the paddle jaw. The traction face 56 can be a contoured portion of the inner surface or an overlay or lamina providing the desired friction with the implantable graft 20. The contours can include a multitude of projections such as individual protrusions or rib structures in any of a variety of patterns. The overlay or lamina can be a material selected to exhibit a sufficiently high coefficient of friction with the implantable graft 20 so as to retain the graft relative to the paddle jaw.

In one configuration, the paddle jaws 52, 54 have an opposing area that is at least 50% of the area of the main body 22 of the implantable graft 20. In selected constructions, the opposing area of the paddle jaws 52, 54 is at least 75% of the area of the main body 22 of the implantable graft 20. In an alternative construction of the paddle jaws 52, 54, the opposing area of the paddle jaws is sized to engage just a sufficient area of the graft 20 to allow for retention and location of the graft without imparting detrimental deformation of the graft. The area of the implantable graft 20 retained between the paddle jaws 52, 54 is selected to stabilize the graft during the procedure.

In one construction of the paddle jaws 52, 54, the outside surface of the paddle jaws are smooth, and in selected configurations configured to generally conform to a portion of a hand of the operator, such as the back of the hand of the operator. Thus, the outside surface can include a contoured recess generally conforming to the back of the hand.

In each configuration of the paddle jaws 52, 54, a front edge 58 of each paddle jaw is rounded to define a rounded leading edge of the graft-retaining clamp 50 in the closed position. The rounding of the front edge is such that in the closed position of the graft-retaining clamp 50, the leading edge has a radius of curvature so as to facilitate movement of the clamp relative to the patient.

It is contemplated the graft-retaining clamp 50 can include a lock mechanism 62 for maintaining the clamp in the closed position (retaining the implantable graft) without requiring an external force by the operator. The lock mechanism 62 is well known in art and can be any of a variety of the known constructions and can be incorporated into the handle shown in FIG. 1.

The tacker guide 70 provides for delivery and presentation of the tacker device 80 to the inside of the finger pocket 26 of the implantable graft 20 and hence the affixing anchor 100 through the graft to the predetermined anatomic location of the patient. The tacker guide 70 includes an elongated cylindrical tube 72 having a proximal end 74 and a distal end 76. An outside dimension of the tube 72 is sized to be simultaneously disposed within the finger pocket 26 of the implantable graft 20 along with a finger tip of the operator. An inner diameter of the tube 72 is sized to permit sliding passage of a portion of the tacker device 80 through the distal end 76. The length of the tacker guide 70 is sufficient to dispose the distal end 76 within the operably located finger pocket 26. It is contemplated the distal end 76 of the tacker guide 70 can be configured to be disposed in a single plane. Alternatively, the distal end 76 can be shaped to provide local regions of increased pressure to assist in maintaining an intended operable position of the distal end. That is, as seen in FIGS. 1A and 1C, the distal end 76 can include a plurality of teeth or projections 78 which engage the graft 20 and reduce movement of the graft prior to subsequent tacking.

The tacker device 80 is used to operably locate the affixing anchor at the predetermined anatomic location and to engage the affixing anchor 100 at the predetermined anatomic location so to affix the implantable graft 20. The tacker device 80 includes an elongate shaft 82 having a handle 84 at a proximal end 86 for manipulation by the operator and a distal end 88 for operable location adjacent the predetermined anatomic location. The handle 84 includes a trigger mechanism 90 operably connected to a delivery head located at the distal end. The delivery head is preloaded to retain at least one affixing anchor 100 so as to present the affixing anchor at the portion of the implantable graft 20 which in turn is at the predetermined anatomic location. That is, the delivery head also locates the affixing anchor 100 with respect to the predetermined anatomic location, thereby disposing a portion of the implantable graft between the affixing anchor and the predetermined anatomic location.

Thus, the tacker device 80 is used to deliver the affixing anchor 100, such as a preloaded spiral corkscrew-shaped absorbable or permanent tacks to affix the implantable graft 20 to the intended point of fixation on a pre-selected anatomic site within the human body (e.g., sacrospinous ligament, levator ani muscles, arcus tendineus fascia pelvis, etc.).

The affixing anchor 100 can be any of a variety of configurations, such as but not limited to staples, rods, tacks, screws, helixes or spirals. In addition, the affixing anchor can be permanent or absorbable as dictated by the intended function of the implantable graft.

The retractor 120 is a shoe-horn shaped device of predetermined dimensions and proportions with one surface 122 being concave and the opposite surface 124 being convex, with a distal end 126 being wider than a proximal end 128. In one configuration, the retractor 120 is formed of a disposable, sterilizable material such as thermoset plastic. However, it is understood any of a variety of known polymer materials can be employed, as are well known in the medical arts. The retractor 120 is sized to support the implantable graft 20 in place, so that any excess material can be truncated during the procedure. Further, the retractor 120 is configured to layout the implantable graft 20 in a smooth substantially tension-free orientation over the corresponding subjacent viscera or anatomic structure, such as the bladder, the bowel or the rectum.

The fenestrated spatula 140 is an elongated rectangular device having a generally measuring tape-like shape, with a first side 142 being concave and a second side 144 being convex. The fenestrated spatula 140 includes a series of fenestrations or apertures 145 along a length of the spatula. In one configuration, the fenestrations 145 are disposed along substantially an entire length of the spatula 140. The fenestrations 145 have a profile sized to cooperate with the distal end 88 of the tacker device 80 to allow passage of the distal end though the fenestration to contact a portion of the implantable graft 20. It has been found satisfactory to form the fenestrations 145 to have an oblong ovoid-shape. The fenestrated spatula 140 allows for the convex side 144 to be oriented downwardly against lateral edges of the implantable graft 20 and permits the placement of the distal end 88 of the tacker device 80 through the pre-shaped fenestrations to affix the lateral edges of the implantable graft to the corresponding predetermined anatomic location (e.g., lateral vaginal/pelvic sidewall) at positions selected at the discretion of the operating surgeon.

It is contemplated each of the graft-retaining clamp 50, the tacker guide 70, the tacker device 80, the retractor 120 and the fenestrated spatula 140 are disposable and formed of a sterilizable material, as known in the art. However, it is understood each of these components could be formed of a metal, alloy or polymer which can withstand sufficient sterilization for reuse.

For illustrative purposes, three procedures are set forth in detail. However, it is understood these three procedures do not limit the scope of the disclosure.

Procedure Description I

Posterior Compartment

A midline incision is made in the epithelium of the posterior vaginal wall from a level just cephalad of the vaginal introitus toward the vaginal apex (or posterior vaginal fornix with a uterus in-situ) stopping short of the actual apex.

Full-thickness vaginal wall flaps are created bilaterally to the level of the pararectal attachments. These rectal “pillars” are further dissected, allowing entry into the ischio-rectal fossae bilaterally. Utilizing blunt digital and sharp dissection, the respective ischial spines are reached and the coccygeus muscle-sacrospinous ligament complex is delineated.

The posterior compartment dissection is completed with detachment of the recto-vaginal fascia from the underside of the vaginal epithelium all the way to the level of the vaginal apex.

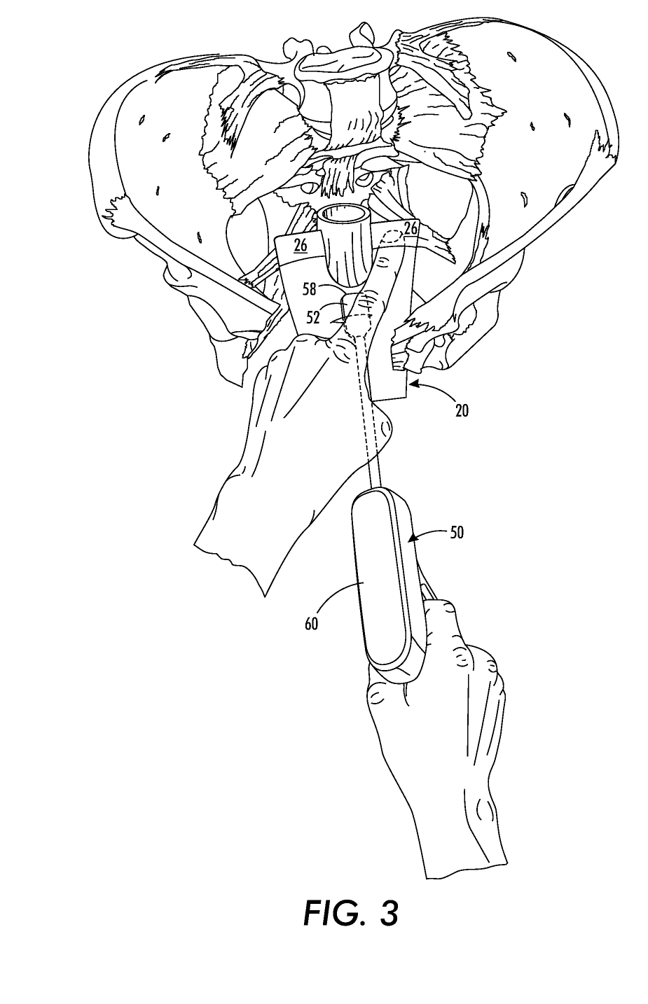

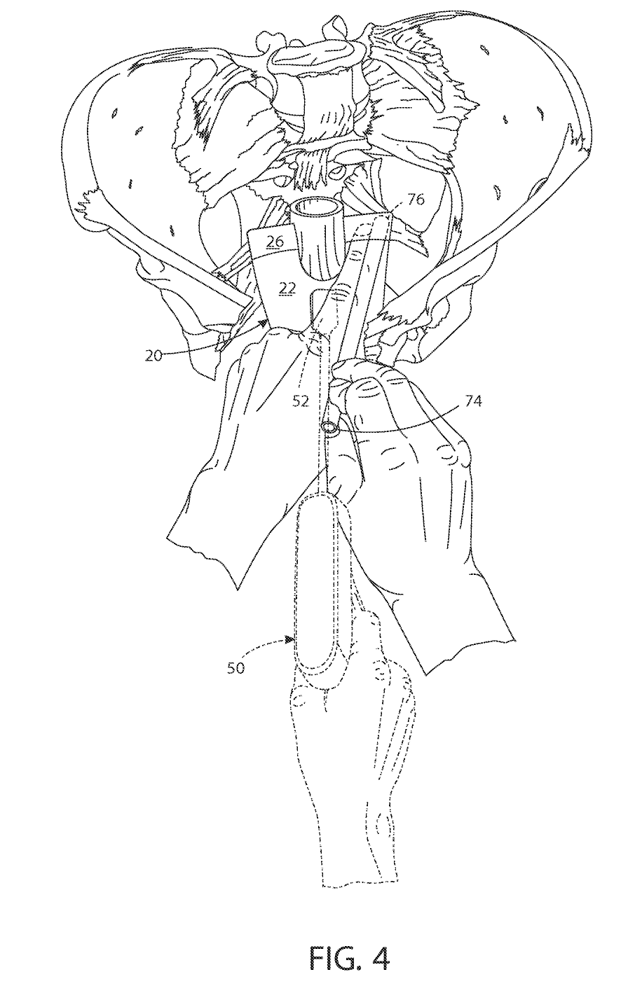

The pre-formed implantable graft 20 is loaded onto the graft-retaining clamp 50 Referring to FIG. 3, the leading edge 58 of the graft-retaining clamp 50 is advanced into the dissected posterior compartment to the apex of the dissection. The graft-retaining clamp 50 is held in this position, as the operator places his/her index finger in the cup-shaped finger pocket 26 of the implantable graft 20. The finger of the operator in the finger pocket 26 is then advanced toward the pre-selected point of anchorage (e.g., sacrospinous ligament or iliococcygeus muscle) and pressed firmly against this structure to provide a point of anchorage. This locates the finger pocket 26, and hence securement appendage 36 if incorporated into the graft at the predetermined anatomic location. As seen in FIG. 4, the tacker guide 70 can be advanced alongside the medial surface of an index finger of the operator (thus protecting the rectum medially). Once the distal end 76 of the tacker guide 70 reaches the level of the point of anchorage (POA), the index finger is pulled back a short distance (approximately 1 cm.). It is also contemplated that the finger of the operator can be disposed below, or outside the finger pocket 26, wherein the tacker guide 70 is advanced into the finger pocket to locate the distal end 76 at the closed end 28 of the finger pocket. The tacker guide 70 is then used press the graft against the intended anatomic location.

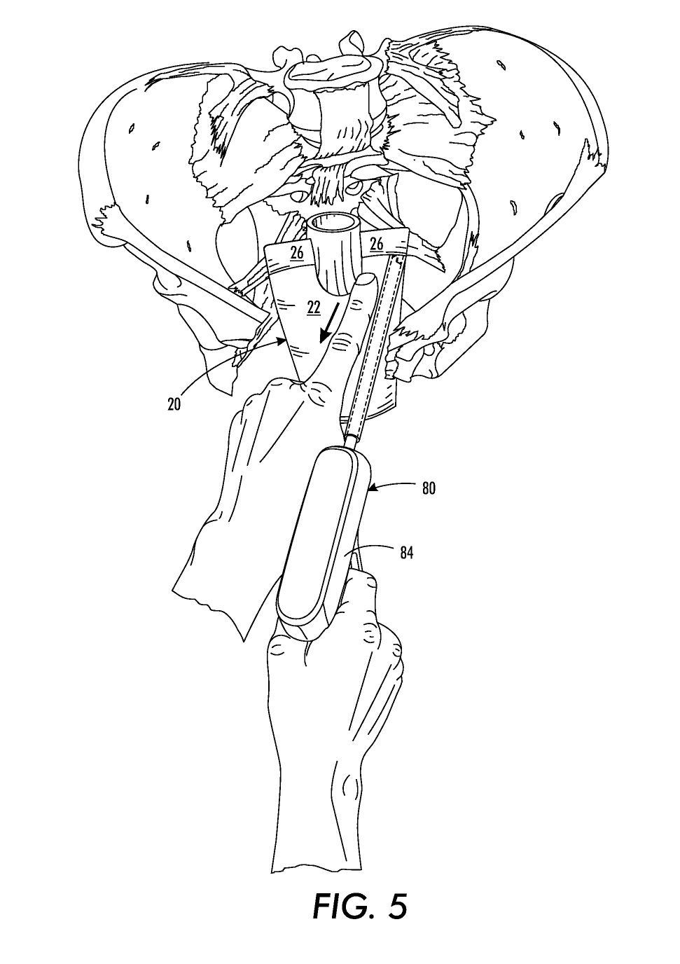

Referring to FIG. 5, the tacker device 80 is passed through the hollow of the tacker guide 70, until the distal end 88 of the tacker device passes through the distal end 76 of the tacker guide and comes into direct contact with the implantable graft 20 overlying the POA. Once the operator has verified appropriate positioning of the implantable graft 20, the tacker guide 70 and the tacker device 80 relative to the POA, the trigger mechanism 90 of the tacker device is activated to release the affixing anchor 100, thus affixing the implantable graft to the POA. It is contemplated the surgeon can withdraw the fingertip from finger pocket 26, while maintaining pressure on the tacker guide 70, so that the intended location of the finger pocket is maintained.

Alternatively, the operator might choose to forego using the tacker guide 70 and simply slide the tacker device 80 alongside his/her finger directly into the finger pocket 26 of the implantable graft 20 and then activate the tacker device to dispose the affixing anchor 100 through the finger pocket and into the POA.

The same maneuver is repeated on a contralateral side of the patient.

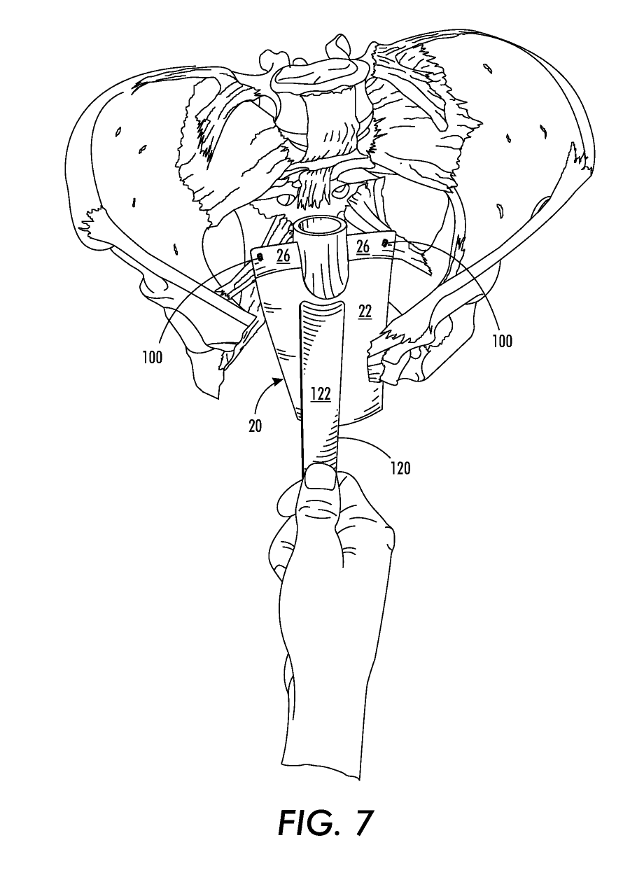

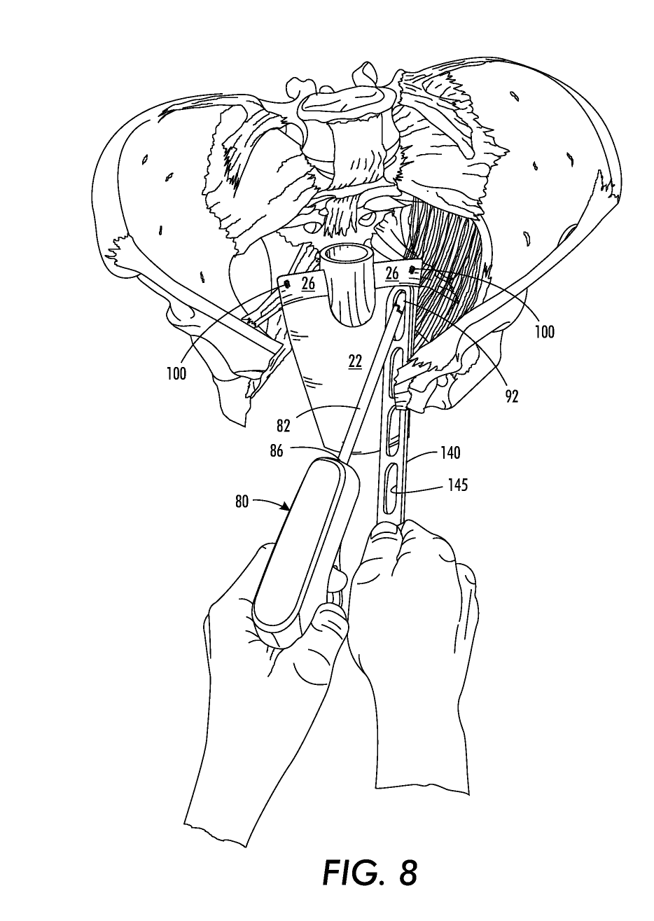

At this point, the implantable graft 20 is released from the graft-retaining clamp 50. Using digital maneuvering, the implantable graft 20 is unfurled to overly the rectum. Referring to FIG. 7, using the shoe-horn shaped retractor 120, the appropriate graft length/width is sized, and the redundant distal portion can be truncated. As seen in FIG. 8, the fenestrated spatula 140 can be positioned over lateral edges of the implantable graft 20. The delivery head of the tacker device 80 is placed through selected fenestrations 145 and the affixing anchor(s) 100 are released. This permits attachment of the posterior graft 20 to the lateral sidewalls. The number of lateral POA's is determined at the discretion of the operator. The most distal point of the graft 20 can be sutured, if necessary.

To reduce bacterial contamination of the implantable graft 20 and operative site, an antibiotic solution can be considered to provide liberal lavage of the entire area throughout the procedure.

Closure of the vaginal incisional site is performed in the usual manner of the operator.

Procedure Description II

Anterior Compartment

A midline vertical incision is made in the epithelium of the anterior vaginal wall from the level of urethrovesical junction (UVJ) toward the vaginal apex (or anterior vaginal fornix with a uterus in-situ) stopping short of the actual apex.

Full-thickness vaginal wall flaps are created bilaterally to the level of the ischiopubic rami. Entry is then accomplished through the endopelvic fascia and into the retropubic space of Retzius. Utilizing blunt digital and sharp dissection, the lateral attachments of the bladder to the pelvic sidewalls (paravaginal attachment to the arcus tendineus fasciae pelvis or ATFP) are separated and dissection proceeds to the level of the respective ischial spines.

The anterior compartment dissection is completed with dissection of the bladder from the underside of the vaginal epithelium all the way to the level of the vaginal apex.

The pre-formed implantable graft 20 for the anterior compartment repair (i.e., anterior graft) is loaded onto the graft-retaining clamp 50. As in the first procedure, leading edge 58 of the graft-retaining clamp 50 is advanced into the dissected anterior compartment to the apex of this dissection in the midline. The graft-retaining clamp 50 is held in this position, as the operator places his/her index finger in the cup-shaped finger pocket 26. The finger pocket 26 is then advanced toward the pre-selected point of anchorage (POA) at the proximal origin of the ATFP near the ischial spine and pressed firmly against this structure. The tacker guide 70 can be advanced alongside the medial surface of the index finger (thus protecting the bladder medially). Once the distal end 76 of the tacker guide 70 reaches the level of the POA, the index finger is pulled back a short distance (approximately 1 cm.). The tacker device 80 is passed through the tacker guide 70, until the delivery head 92 reaches the distal end 76 and comes into direct contact with the implantable graft 20 overlying the point of POA. Once the operator has verified appropriate positioning of the implantable graft 20, the tacker guide 70 and the tacker device 80 relative to the POA, the trigger mechanism 90 is activated to release the affixing anchor 100, thus affixing the implantable graft to the POA.

Alternatively, the operator might choose to forego using the tacker guide 70 and simply slide the tacker device 80 alongside his/her finger directly onto the implantable graft 20 and then engage the affixing anchor 100 to the POA. The same maneuver is repeated on the contralateral side of the patient.

At this point, the implantable graft 20 is released from the graft-retaining clamp 50. Using digital maneuvering, the implantable graft 20 is unfurled beneath the bladder base. Using the shoe-horn shaped retractor 120, the appropriate graft length is sized, and any redundant distal portion is truncated at the discretion of the surgeon. The fenestrated spatula 140 is placed along the lateral edges of the graft 20. The distal end 88 of the tacker device 80 is passed through selected fenestrations 145 and the affixing anchors 100 are released. This permits attachment of the anterior graft 20 to the lateral sidewalls in the proximity of the so-called White line or arcus tendineus fasciae pelvis. The number of lateral points of attachment are determined at the discretion of the operator. The most distal point of the graft can be sutured near the urethrovesical junction, if necessary.

To reduce bacterial contamination of the graft 20 and operative site an antibiotic solution needs to be considered to provide liberal lavage of the entire area throughout the procedure.

Closure of the vaginal incisional site is performed in the usual manner of the operator.

Procedure Description III

Combined Anterior/Posterior Compartments

Concomitant resuspensions of the anterior and posterior compartments can be accomplished by combining the above-detailed procedural steps and utilizing the aforementioned instruments and grafts.

Procedure Description IV

Apical Compartment

Resuspension of a prolapsing vaginal apical compartment can be accomplished by preferentially suspending one compartment first (either posterior or anterior) and then suspending the opposite compartment. During the suspension of the latter compartment, the uppermost portions of the two graft components (i.e., anterior, posterior) can be sutured together, with subsequent closure of the vaginal incision(s). These procedural steps will require the expertise and discretionary improvisation of the operating surgeon to attain the objective of resuspending any and/or all three vaginal compartments.

In the case of resuspending a prolapsing uterus as part of the vaginal reconstructive surgery, the corresponding graft 20 is affixed to the cervical stroma with non-absorbable permanent sutures, prior to affixing the graft to the POA of the corresponding compartment, as detailed above. In this fashion, the uterus and corresponding compartment are elevated and/or suspended concurrently.

The present system can provide increased convenience and safety for surgeons with the attendant advantages for the patient. The present system can also take advantage of existing surgeon training and preferences to potentially contribute to better surgical outcomes. Surgical procedures utilizing the present system have the capacity to be shorter in duration, easier to perform and potentially safer and more reliable with concomitant advantages for patients and the healthcare system.

The foregoing embodiments of the invention are representative embodiments, and are provided for illustrative purposes. The embodiments are not intended to limit the scope of the invention. Variations and modifications are apparent from a reading of the preceding description and are included within the scope of the invention. The invention is intended to be limited only by the scope of the accompanying claims.

1. A method of retaining an implantable graft relative to a patient, the method comprising:
(a) passing an affixing anchor through a finger pocket of the implantable graft to pass the affixing anchor into an anatomic location of the patient to dispose a portion of the implantable graft intermediate the affixing anchor and the patient, wherein the finger pocket is sized to receive a fingertip of an operator.

2. The method of claim 1, further comprising forming the implantable graft to have a main body and integrally forming the finger pocket with the main body.

3. The method of claim 1, further comprising forming the affixing anchor to include a spiral shape.

4. The method of claim 1, further comprising forming the affixing anchor to include a helical shape.

5. The method of claim 1, further comprising passing the affixing anchor through a portion of one of the sacrospinous ligament and an adjacent anatomic location.

6. The method of claim 1, further comprising releasably retaining a portion of the implantable graft in a graft-retaining clamp prior to passing the affixing anchor through a portion of the implantable graft.

7. The method of claim 1, further comprising employing a mesh as the implantable graft.

8. The method of claim 1, further comprising forming the finger pocket integral with a securement appendage, the securement appendage extending from a main body of the implantable graft.

9. The method of claim 1, further comprising inserting an operator fingertip into the finger pocket to locate the finger pocket adjacent to the anatomic portion of the patient prior to passing the affixing anchor through the finger pocket.

10. The method of claim 1, further comprising inserting an operator fingertip into a second finger pocket to locate the second finger pocket adjacent to a second anatomic structure of the patient.

11. The method of claim 1, further comprising passing the affixing anchor through a tacker guide prior to passing the affixing anchor through the portion of the implantable graft.

12. An implantable graft comprising:
(a) a main body formed of a biocompatible material, the main body including a finger pocket, the finger pocket sized to engage an operator fingertip and at least one securement appendage extending from the main body, the securement appendage extending from the main body along securement appendage axis intersecting the longitudinal axis, the finger pocket being fixedly attached to the securement appendage.

13. (canceled)

14. The implantable graft of claim 12, wherein the finger pocket is integral with the securement appendage.

15. The implantable graft of claim 12, further comprising a second securement appendage extending from the main body along a second securement appendage axis intersecting the longitudinal axis, the second securement appendage including a second finger pocket spaced from the main body, the second finger pocket sized to engage a fingertip.

16. The implantable graft of claim 12, wherein the main body includes an anterior portion and a posterior portion separated by a fold line.

17. An apparatus for temporarily retaining an implantable mesh graft, the apparatus comprising:
(a) an implantable graft having a finger pocket; and
(b) a graft-retaining clamp having a first paddle jaw and a second opposing paddle jaw, the paddle jaws moveable between an open position and a closed position, an inner surface of at least one of the first and the second paddle jaw including a traction face for releasable engaging the implantable mesh graft and an outer surface of the at least one of the first and the second paddle jaw including a contoured recess, the contoured recess sized to cooperatively receive a portion of a back of a hand of an operator.

18. The apparatus of claim 17, wherein an inner surface of each of the first and the second paddle jaw includes a traction face for releasable engaging the implantable graft.

19. (canceled)

20. A method of connecting an implantable graft to a patient, the method comprising:
(a) inserting a tacker device into a pocket formed on an implantable graft to locate an outer surface of the pocket against a predetermined anatomic location of the patient; and
(b) passing an affixing anchor through the pocket into the patient to dispose a portion of the implantable graft intermediate the affixing anchor and the patient.

21. A surgical kit comprising:
(a) a sterilizable container;
(b) an implantable graft within the container, the implantable graft having a finger pocket sized to receive an operator fingertip; and
(c) a substantially cylindrical tacker guide within the container, the tacker guide having a proximal end and a distal end, the distal end sized to be disposed within the finger pocket simultaneous with an operator fingertip.

22. The surgical kit of claim 21, further comprising a graft-retaining clamp having opposing paddle jaws, the paddle jaws sized to retain a portion of the implantable graft therebetween.

23. The surgical kit of claim 21, further comprising a fenestrated spatula in the container.
